(19) United States
(12) Patent Application Publication
(10) Pub. No.: US 2023/0270748 A1
(43) Pub. Date: Aug. 31, 2023

(54) COMBINATION OF A BCL-2 INHIBITOR AND A HYPOMETHYLATING AGENT FOR TREATING CANCERS, USES AND PHARMACEUTICAL COMPOSITIONS THEREOF

(71) Applicants: LES LABORATOIRES SERVIER, SURESNES CEDEX (FR); NOVARTIS AG, Basel (CH)

(72) Inventors: Emmelyne DESSEIN, RUEIL MALMAISON (FR); Lisa MAHNKE, Boston, MA (US); Michael Robert COOPER, Sudbury, MA (US)

(21) Appl. No.: 18/007,321

(22) PCT Filed: Jul. 30, 2021
(86) PCT No.: PCT/EP2021/071368

§ 371 Date: Jan. 30, 2023

Related U.S. Application Data
(60) Provisional application No. 63/059,419, filed on Jul. 31, 2020.

(30) Foreign Application Priority Data
Sep. 11, 2020 (EP) 20195633.1

(51) Int. Cl. A61K 31/5377; A61K 31/706

(52) U.S. Cl. CPC A61K 31/5377; A61K 31/706

(57) ABSTRACT

A combination comprising a Bcl-2 inhibitor with a hypomethylating agent, uses in the treatment of cancers and pharmaceutical compositions thereof. The Bcl-2 inhibitor is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide and the hypomethylating agent is selected from decitabine, azacitidine and guadecitabine.

### FIELD OF THE INVENTION

The present invention relates to the combination of a Bcl-2 inhibitor with a hypomethylating agent (HMA) selected from decitabine, azacitidine and guadecitabine, more particularly azacitidine. The Bcl-2 inhibitor is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide, referred to herein as ‘Compound A’, or a pharmaceutically acceptable salt thereof. The invention also relates to the use of said combination in the treatment of cancer, in particular haematological malignancies, and more particularly acute myeloid leukemia (AML), myelodysplastic syndromes (MDS), lymphoma, chronic lymphocytic leukemia (CLL) and multiple myeloma. Also provided are pharmaceutical formulations suitable for the administration of such combinations. ‘Compound A’ as used herein optionally includes the pharmaceutically acceptable salts thereof.

The presence of multiple acquired mutations within multiple clones in each AML case makes the concept of successful selective targeting particularly difficult. This invention proposes the concept that cancers with diverse and multi-clonal molecular compositions may be successfully treated with the combination of an inhibitor of Bcl-2 and a cytotoxic drug able to effectively activate cellular apoptosis in a promiscuous manner, thereby leading to broad-based cell death of cancer cells beyond that achieved using the Bcl-2 inhibitor or the hypomethylating agent separately. This approach may lead to reduced rates of disease relapse and higher overall cure rates in AML as an example. AML is proposed as a model example due to the ability to quantitatively measure changes in clonal composition serially with treatment using digital PCR and RT-qPCR.

### BACKGROUND OF THE INVENTION

Apoptosis is a highly regulated cell death pathway that is initiated by various cytotoxic stimuli, including oncogenic stress and chemotherapeutic agents. It has been shown that evasion of apoptosis is a hallmark of cancer and that efficacy of many chemotherapeutic agents is dependent upon the activation of the intrinsic mitochondrial pathway. Three distinct subgroups of the Bcl-2 family proteins control the intrinsic apoptosis pathway:

(i) the pro-apoptotic BH3 (the Bcl-2 homology 3)-only proteins; (ii) the pro-survival members such as Bcl-2 itself, Bcl-xl, Bcl-w, Mcl-1 and Bcl-2a1; and (iii) the pro-apoptotic effector proteins BAX and BAK (Czabotar et al., Nature Reviews Molecular Cell Biology 2014, 15, 49-63). Overexpression of the anti-apoptotic members of Bcl-2 family is observed in many cancers, particularly in hematological malignancies such as mantle cell lymphoma (MCL), follicular lymphoma/diffuse large B-cell lymphoma (FL/DLCL) and multiple myeloma (Adams and Cory, Oncogene 2007, 26, 1324-1337). Pharmacological inhibition of the anti-apoptotic proteins Bcl-2, Bcl-xl, Bcl-w and Mcl-1 by the recently developed BH3-mimetics drugs such as ABT-199 (venetoclax), ABT-263 (navitoclax), S55746/BCL201 and S63845 has emerged as a therapeutic strategy to induce apoptosis and cause tumor regression in cancer (Zhang et al., Drug Resist. Updat. 2007, 10, 207-217; Casara et al, Oncotarget 2018, Vol. 9, No. 28, 20075-20088 and corresponding Supplementary Information; Kotschy et al., Nature 2016, 538, 477-482). Nevertheless, mechanisms of resistance to BH3 mimetics have been observed (Choudhary et al., Cell Death and Disease 2015, 6, e1593) and the use of combination therapies could improve efficacy and delay or even abrogate resistance development.

Acute myeloid leukemia (AML) is a rapidly fatal blood cancer arising from clonal transformation of hematopoietic stem cells resulting in paralysis of normal bone marrow function and deaths due to complications from profound pancytopenia. AML accounts for 25% of all adult leukemias, with the highest incidence rates occurring in the United States, Australia and Europe (WHO. GLOBOCAN 2012. Estimated cancer incidence, mortality and prevalence worldwide in 2012. International Agency for Research on Cancer). Globally, there are approximately 88,000 new cases diagnosed annually. AML continues to have the lowest survival rate of all leukemias, with expected 5-year survival of only 26.9%.

Untreated patients succumb to AML within weeks (Institute NC. Cancer Stat Facts: AML, 2017). The incidence of AML increases approximately by 10-fold with age, from 1.3 cases per 100,000 people under 65 years of age to 12.2 cases per 100,000 in those over 65 years of age (De Kouchkowsky et al, Blood Cancer J. 2016; 6(7):e44). Older patients present a challenge in the management of AML. In this population, AML is characterized by unfavourable karyotypes and higher mutational burden. Furthermore, the incidence of secondary AML, related to prior MDS or prior chemotherapy is higher in this population where current standard-of-care regimen may be difficult to tolerate, and treatment-related mortality may exceed expected anti-leukemic response. Due to the high rates of relapse or recurrence in AML, there is a great need for effective first line therapies.

Current intensive therapies for the treatment of AML include the administration of cytarabine alone or in combination with an anthracycline such as daunorubicin or idarubicin. Low-dose cytarabine treatment and demethylating agents such as azacitidine and decitabine are also recommended as low-intensity options for patients who are ineligible for intensive chemotherapy (Döhner et al., DOI 10.1182/blood-2016-08-733196). Although the standard therapy for AML (cytarabine in combination with anthracyclines) was conceived over 4 decades ago, the introduction of successful targeted therapies for this disease has remained an elusive goal. The concept of targeted therapy in AML has been hampered by the realization that this disease evolves as a multi-clonal hierarchy, with rapid outgrowth of leukemic sub-clones as a major cause of drug resistance and disease relapse (Ding et al., Nature 2012, 481, 506-510). Recent clinical investigations have demonstrated the efficacy of Bcl-2 inhibitors in the treatment of AML (Konopleva et al., American Society of Hematology 2014, 118).

In recent years, much has been learned about the genomic and epigenomic landscapes of AML, and the clonal architecture of both de novo and secondary AML has begun to be unraveled. New targeted therapies are in development or have been approved by the US Food and Drug Administration (FDA) and/or European Medicine Agency (EMA) for treatment of AML. Indeed, the FLT3 inhibitors midostaurin and gilteritinib, the antibody-drug conjugate gemtuzumab ozogamicin, CPX-351 (liposomal daunorubicin and cytarabine), the IDH2 inhibitor enasidenib, IDH1 inhibitor ivosidenib, the Hedghog pathway inhibitor glasdegid, and the Bcl-2 inhibitor venetoclax were approved. In 2018, venetoclax got an accelerated approval in US based on phase I/II results (based on complete response rate) in first line unfit AML patients not eligible for intensive chemotherapy in combination with HMA (DiNardo et al, Am J Hematol. 2018; 93:401-407; DiNardo et al, Lancet. 2018; 19 216-228; Wei et al, J Clin Oncol. 2019; 37:1277-1284). In June 2020, the results of the confirmatory phase III study showed that the venetoclax in combo with HMA reduced the risk of death (overall survival [OS]) by 34% compared to azacitidine alone in patients with previously untreated AML. Venetoclax plus azacitidine combination also led to higher rates of composite complete response (CR+CR with incomplete blood count recovery [CR+CRi]) at 66.4% compared to 28.3% with azacitidine alone (p<0.001) (DiNardo et al, Abstract presented at EHA congress, June 2020).

Despite advances in our understanding of the molecular basis of AML subtypes and new targeted therapies approved for patients with FLT3 mutant, IDH2 mutant, CD33 positive AML, t-AML, and AML-MRC (Wei et al, Blood 2017; 130(23): 2469-2474), a significant proportion of patients with AML has limited treatment options. New therapies are clearly needed, and in particular, there is a need to develop therapy combinations that avoid the use of cytotoxic drugs.

More globally, there remains a need for new treatments and therapies for the treatment of a haematological malignancies, including AML as discussed supra, myelodysplastic syndromes, lymphoma, chronic lymphocytic leukemia and multiple myeloma. In this context, the present invention provides a novel combination of a Bcl-2 inhibitor, namely Compound A, with a hypomethylating agent selected from decitabine, azacitidine and guadecitabine, and more preferably azacitidine.

The structure of Compound A is:

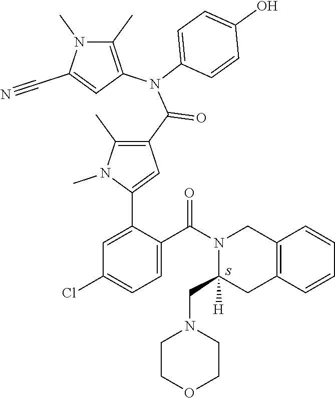

5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide. The preparation of Compound A, its use as a Bcl-2 inhibitor for the treatment of cancer and pharmaceutical formulations thereof, are described in WO 2015/011400, the content of which is incorporated by reference. The preparation is specifically disclosed in Example 386 of WO 2015/011400 in the form of a hydrochloride salt and hydrogen sulfate salt of the same is also described in WO 2020/089281. Furthermore, cyclodextrin-based formulations comprising Compound A are shown in WO 2020/089286.

The results show that Compound A, the Bcl-2 inhibitor according to the invention, and azacitidine interact synergistically in AML cell lines (FIG. 1-4; Table 2).

### SUMMARY OF THE INVENTION

The present invention relates to a combination comprising:
(a) a Bcl-2 inhibitor which is ‘Compound A’ and,
(b) a hypomethylating agent selected from decitabine, azacitidine and guadecitabine, for simultaneous, sequential or separate use.

In a preferred embodiment, the hypomethylating agent is azacitidine.

In another embodiment, the Compound A is administered parenterally. In particular, such administration is by intravenous infusion in order to achieve higher exposure and decrease inter-patient exposure variability compared to an oral administration.

In a specific embodiment, Compound A is administered once a week. Such schedule of administration may be optimal in terms of effectiveness-tolerability profile by assuming that the efficacy of Compound A is driven by Cₘₐₓ, as supported by preclinical observations. Preferably, azacitidine will be administered according to a 5-2-2 schedule during a 28-day cycle as follows:
5-consecutive days (D1-D5) followed by a 2-day break (D6-D7) and then for 2 days (D8-D9),
followed by a rest period of 19 days.

Such 5-2-2 azacitidine regimen (instead of 7-0 regimen recommended by labelling) allows an important overlap of exposure of both drugs as a co-administration of Compound A and azacitidine will occur on D1 and D8 of each cycle. This co-exposure is expected to increase the activity of combination therapy since preclinical synergy is observed between the two agents.

In another embodiment, Compound A is administered on day 1 (D1), day 3 (D3), day 5 (D5) and day 8 (D8) in the two first weeks of the cycle, wherein the single doses administered on D1, D3, D5 and D8 are identical to each other. In a further embodiment, Compound A is administered on day 1 (D1), day 2 (D2), day 3 (D3), day 4 (D4), day 5 (D5), day 8 (D8) and day 9 (D9) in the two first weeks of the cycle, wherein the single doses administered on D1, D2, D3, D4, D5, D8 and D9 are identical to each other. Preferably, azacitidine is administered according to a 5-2-2 schedule during a 28-day cycle as detailed previously. These administration schedules aims to maximize the potential for synergistic anti tumor activity between azacitidine and Compound A by increasing the overlap of exposure at all cycles during the first week.

Moreover, the different peak-to-trough ratio and longer time of undetectable concentrations of Compound A compared to daily dosed drugs such as venetoclax means there is expected better effect on bone marrow in particular, which needs time to recover and which is a limiting factor for consistent dosing of venetoclax. If cytopenia (including neutropenia) is improved relative to venetoclax, then indications with particular sensitivity to infectious complications such as AML and multiple myeloma may be more tractable for patients treated with Compound A and azacytidine.

In another embodiment, the invention provides a combination as described herein, for use in the treatment of cancer, more particularly, the treatment of haematological malignancies. The treatment of AML, myelodysplastic syndromes, lymphoma and multiple myeloma is particularly preferred. More particularly, the treatment of AML is targeted.

### BRIEF DESCRIPTION OF THE FIGURES

FIG. 1 illustrates an exemplary cell growth inhibition effect and synergy combination matrices for inhibition of cell growth (left) and Loewe excess inhibition (right) afforded by Compound A (Bcl-2 inhibitor) in combination with azacitidine in the AML cell line OCI-AML3 in two independent experiments. Values in the dose matrix range from 0 (no inhibition) to 100 (total inhibition). Values in the Loewe excess matrix represent the extent of growth inhibition in excess of the theoretical additivity calculated based on the single agent activities of Compound A and azacitidine at the concentrations tested.

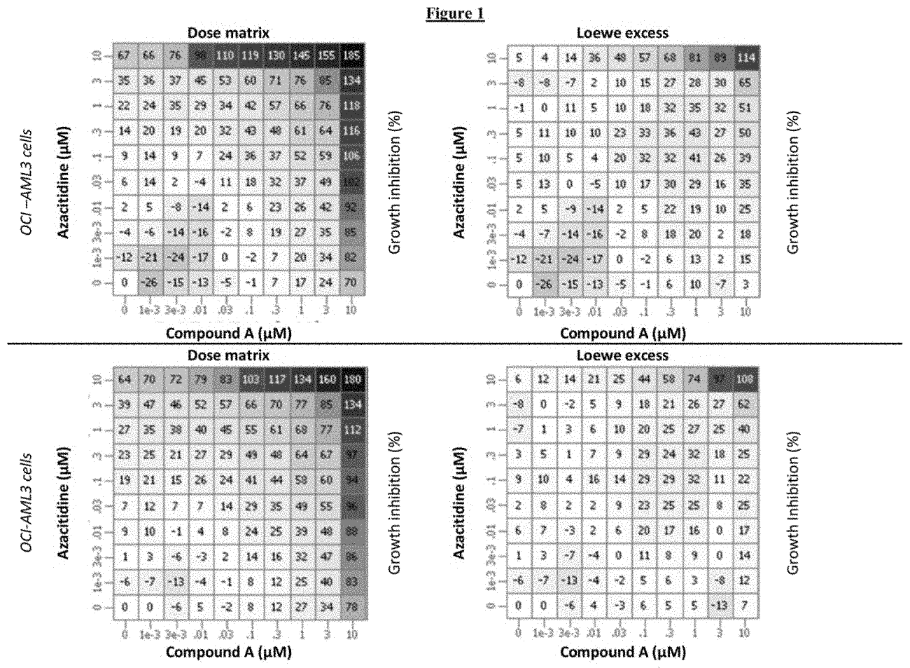

FIG. 2 illustrates an exemplary cell growth inhibition effect and synergy combination matrices for inhibition of cell growth (left) and Loewe excess inhibition (right) afforded by Compound A (Bcl-2 inhibitor) in combination with azacitidine in the AML cell line HL-60 in two independent experiments.

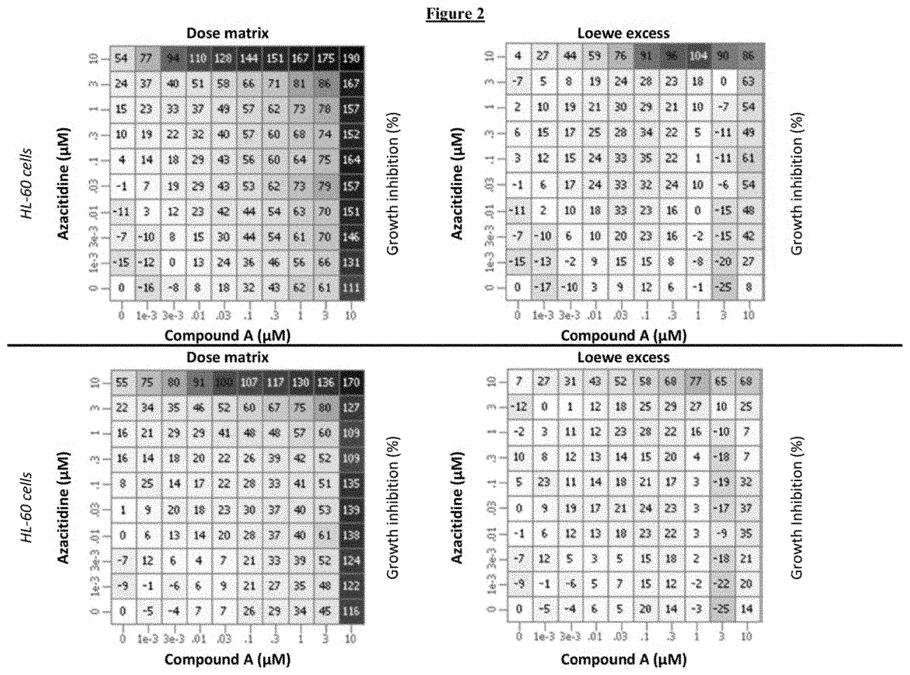

FIG. 3 illustrates an exemplary cell growth inhibition effect and synergy combination matrices for inhibition of cell growth (left) and Loewe excess inhibition (right) afforded by Compound A (Bcl-2 inhibitor) in combination with azacitidine in the AML cell line MV4;11 in two independent experiments.

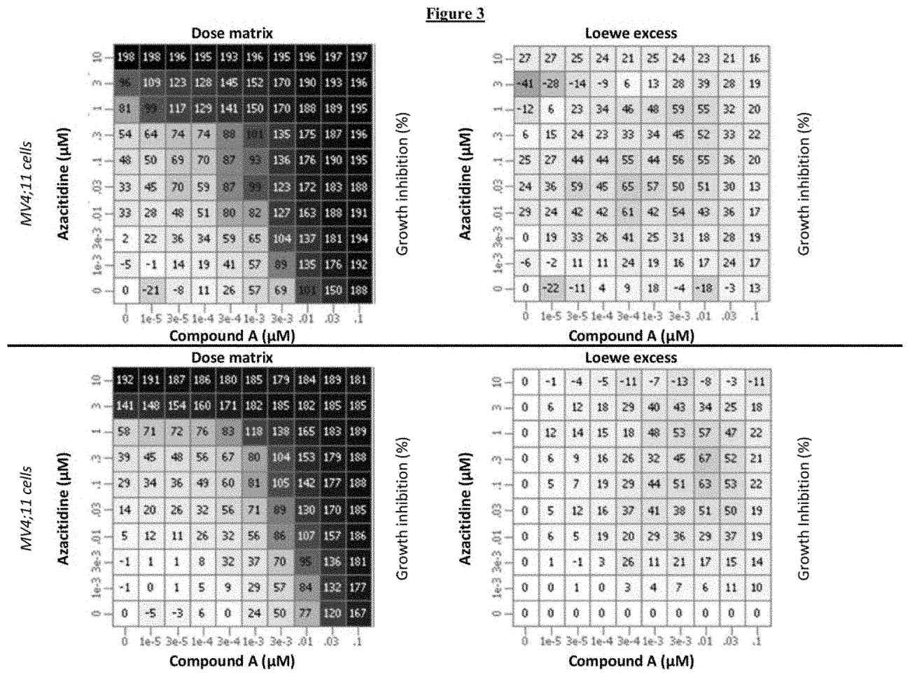

FIG. 4 illustrates an exemplary cell growth inhibition effect and synergy combination matrices for inhibition of cell growth (left) and Loewe excess inhibition (right) afforded by Compound A (Bcl-2 inhibitor) in combination with azacitidine in the AML cell line EOL-1 in two independent experiments.

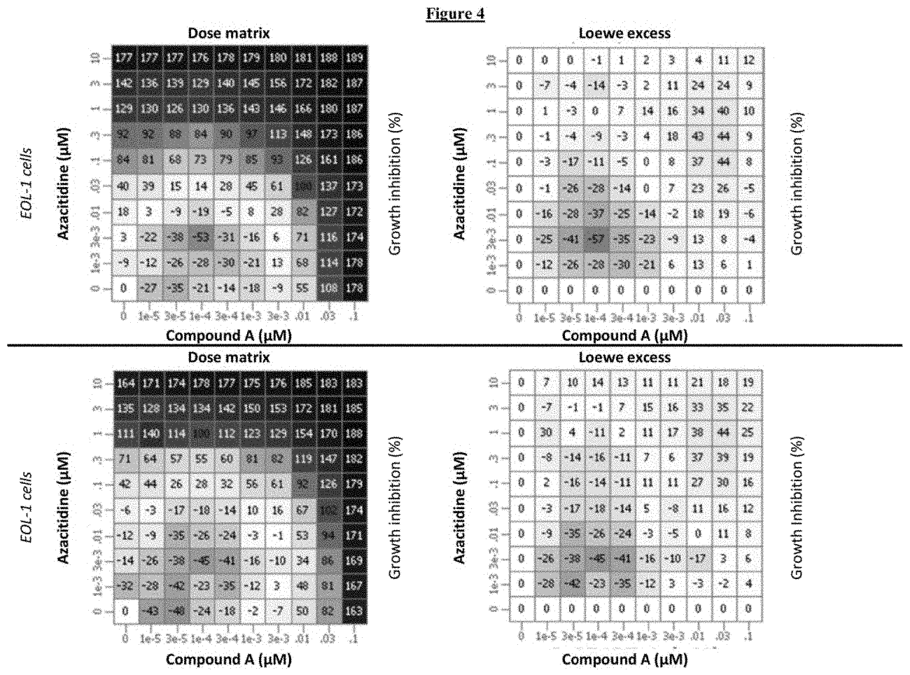

### DETAILED DESCRIPTION OF THE INVENTION

The invention therefore provides in Embodiment E1, a combination comprising:
(a) a Bcl-2 inhibitor which is (‘Compound A’):

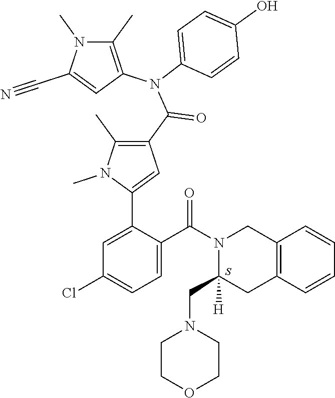

and
(b) a hypomethylating agent selected from decitabine, azacitidine and guadecitabine, for simultaneous, sequential or separate use.

E2. The combination according to E1 wherein the hypomethylating agent is azacitidine.

E3. The combination according to E1 or E2, wherein Compound A is in the form of a hydrogen sulfate salt.

E4. The combination according to any one of E1 to E3, for use in the treatment of cancer.

E5. The combination for use according to E4 wherein the cancer is a haematological malignancy.

E6. The combination for use according to E5 wherein the haematological malignancy is acute myeloid leukemia (AML).

E7. The combination for use according to E5 wherein the haematological malignancy is myelodysplastic syndromes.

E8. The combination for use according to E5 wherein the haematological malignancy is lymphoma.

E9. The combination for use according to E5 wherein the haematological malignancy is chronic lymphocytic leukemia.

E10. The combination for use according to E5 wherein the haematological malignancy is multiple myeloma.

E11. The combination for use according to any one of E4 to E10, wherein Compound A and the hypomethylating agent are provided in amounts which are jointly therapeutically effective for the treatment of cancer.

E12. The combination for use according to any one of E4 to E10, wherein Compound A and the hypomethylating agent are provided in amounts which are synergistically effective for the treatment of cancer.

E13. The combination for use according to E12, wherein the Compound A and the hypomethylating agent are provided in synergistically effective amounts which enable a reduction of the dose required for each compound in the treatment of cancer, whilst providing an efficacious cancer treatment, with eventually a reduction in side effects.

E14. The combination according to any of E4 to E13, wherein Compound A is administered parentally, more particularly intravenously.

E15. The combination according to E14, wherein the dose of Compound A per administration is from 25 mg to 500 mg. In another embodiment, the dose of Compound A per administration is from 25 to 1000 mg or from 25 to 1500 mg.

E16. The combination according to E15, wherein Compound A is administered once a week.

E17. The combination according to E16 wherein Compound A and azacitidine are administered during a 28-day cycle as follows:
(i) Compound A is administered on day 1 (D1), day 8 (D8), day 15 (D15) and day 22 (D22) and,
(ii) azacitidine is administered according to a 5-2-2 schedule:
   5-consecutive days (D1-D5) followed by a 2-day break (D6-D7) and then for 2 days (D8-D9),
   followed by a rest period of 19 days.

E18. The combination according to E14 wherein Compound A and azacitidine are administered during a 28-day cycle as follows:
(i) Compound A is administered on day 1 (D1), day 3 (D3), day 5 (D5) and day 8 (D8) in the two first weeks of the cycle, wherein the single doses administered on D1, D3, D5 and D8 are identical to each other;
(ii) azacitidine is administered according to a 5-2-2 schedule:
   5-consecutive days (D1-D5) followed by a 2-day break (D6-D7) and then for 2 days (D8-D9),
   followed by a rest period of 19 days.

E19. The combination according to E14 wherein Compound A and azacitidine are administered during a 28-day cycle as follows:
(i) Compound A is administered on day 1 (D1), day 2 (D2), day 3 (D3), day 4 (D4), day 5 (D5), day 8 (D8) and day 9 (D9) in the two first weeks of the cycle, wherein the single doses administered on D1, D2, D3, D4, D5, D8 and D9 are identical to each other;
(ii) azacitidine is administered according to a 5-2-2 schedule:
   5-consecutive days (D1-D5) followed by a 2-day break (D6-D7) and then for 2 days (D8-D9),
   followed by a rest period of 19 days.

E20. The combination according to any one of E1 to E3, further comprising one or more excipients.

E21. The use of a combination according to any one of E1 to E3, in the manufacture of a medicament for the treatment of cancer.

E22. The use according to E21 wherein the cancer is a haematological malignancy.

E23. The use according to E22 wherein the haematological malignancy is acute myeloid leukemia (AML).

E24. The use according to E22 wherein the haematological malignancy is myelodysplastic syndromes.

E25. The use according to E22 wherein the haematological malignancy is lymphoma.

E26. The use according to E22 wherein the haematological malignancy is chronic lymphocytic leukemia.

E27. The use according to E22 wherein the haematological malignancy is multiple myeloma.

E28. A medicament containing, separately or together,
(a) a Bcl-2 inhibitor which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (‘Compound A’):

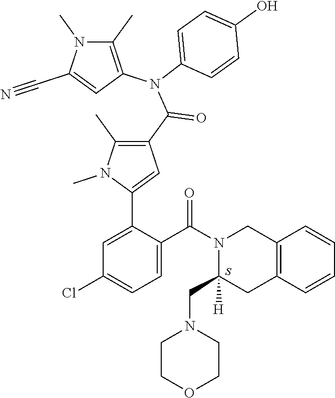

and
(b) a hypomethylating agent, preferably azacitidine,
for simultaneous, sequential or separate administration, and wherein the Compound A and the hypomethylating agent are provided in effective amounts for the treatment of cancer.

E29. A method of treating cancer, comprising administering a jointly therapeutically effective amount of:
(a) a Bcl-2 inhibitor which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (‘Compound A’):

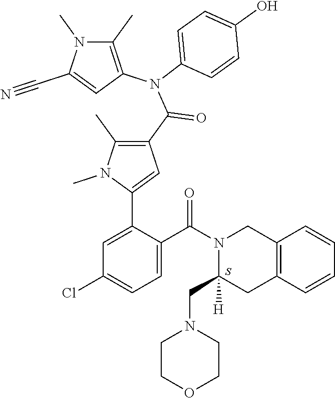

and
(b) a hypomethylating agent,
to a subject in need thereof.

E30. A method for sensitizing a patient who is (i) refractory to at least one chemotherapy treatment, or (ii) in relapse after treatment with chemotherapy, or both (i) and (ii), wherein the method comprises administering a jointly therapeutically effective amount of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (‘Compound A’):

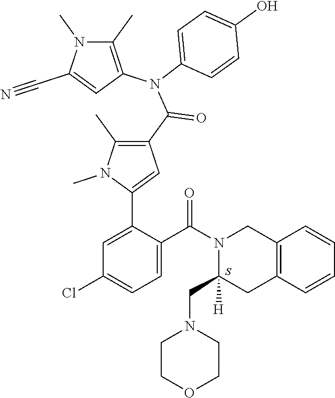

in combination with a hypomethylating agent to said patient.

E31. The method according to E29 or E30 wherein the hypomethylating agent is azacitidine.

E32. The method according to E31 wherein Compound A and azacitidine are administered during a 28-day cycle as follows:
(i) Compound A is administered on day 1 (D1), day 8 (D8), day 15 (D15) and day 22 (D22) and,
(ii) azacitidine is administered according to a 5-2-2 schedule:
   5-consecutive days (D1-D5) followed by a 2-day break (D6-D7) and then for 2 days (D8-D9),
   followed by a rest period of 19 days.

E33. The method according to E31 wherein Compound A and azacitidine are administered during a 28-day cycle as follows:
(iii) Compound A is administered on day 1 (D1), day 3 (D3), day 5 (D5) and day 8 (D8) in the two first weeks of the cycle, wherein the single doses administered on D1, D3, D5 and D8 are identical to each other;
(iv) azacitidine is administered according to a 5-2-2 schedule:
   5-consecutive days (D1-D5) followed by a 2-day break (D6-D7) and then for 2 days (D8-D9),
   followed by a rest period of 19 days.

E34. The method according to E31 wherein Compound A and azacitidine are administered during a 28-day cycle as follows:
(iii) Compound A is administered on day 1 (D1), day 2 (D2), day 3 (D3), day 4 (D4), day 5 (D5), day 8 (D8) and day 9 (D9) in the two first weeks of the cycle, wherein the single doses administered on D1, D2, D3, D4, D5, D8 and D9 are identical to each other;
(iv) azacitidine is administered according to a 5-2-2 schedule:
   5-consecutive days (D1-D5) followed by a 2-day break (D6-D7) and then for 2 days (D8-D9),
   followed by a rest period of 19 days.

‘Compound A’ means 5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide. ‘Compound A’ as used herein optionally includes the pharmaceutically acceptable salts thereof.

‘Compound A, H₂SO₄’ means that 5-(5-chloro-2-{[(3S)-3-(morpholin-4-ylmethyl)-3,4-dihydroisoquinolin-2(1H)-yl]carbonyl} phenyl)-N-(5-cyano-1,2-dimethyl-1H-pyrrol-3-yl)-N-(4-hydroxyphenyl)-1,2-dimethyl-1H-pyrrole-3-carboxamide is in the form of a hydrogen sulfate salt.

‘Free molecule’ and ‘free base’ are used interchangeably herein and refer to Compound A when not in salt form.

‘Combination’ refers to either a fixed dose combination in one unit dosage form (e.g., capsule, tablet, or sachet), non-fixed dose combination, or a kit of parts for the combined administration where Compound A and one or more combination partners (e.g. another drug as explained below, also referred to as ‘therapeutic agent’ or ‘co-agent’) may be administered independently at the same time or separately within time intervals, especially where these time intervals allow that the combination partners show a cooperative, e.g. synergistic effect.

The terms ‘co-administration’ or ‘combined administration’ or the like as utilized herein are meant to encompass administration of the selected combination partner to a single subject in need thereof (e.g. a patient), and are intended to include treatment regimens in which the agents are not necessarily administered by the same route of administration or at the same time.

The term ‘fixed dose combination’ means that the active ingredients, e.g. Compound A and one or more combination partners, are both administered to a patient simultaneously in the form of a single entity or dosage.

The term ‘non-fixed dose combination’ means that the active ingredients, e.g. Compound A and one or more combination partners, are both administered to a patient as separate entities either simultaneously or sequentially, with no specific time limits, wherein such administration provides therapeutically effective levels of the two compounds in the body of the patient. The latter also applies to cocktail therapy, e.g. the administration of three or more active ingredients.

‘Cancer’ means a class of disease in which a group of cells display uncontrolled growth. Among the cancer treatments envisaged there may be mentioned, without implying any limitation, the treatment of haematological malignancies and solid tumors. Haematological malignancies include myeloma, especially multiple myeloma, lymphoma, especially Non-Hodgkin Lymphoma (NHL) and more especially Diffuse Large B-cell Lymphoma (DLBCL), and leukemia, especially Chronic Lymphocytic Leukemia (CLL), T-cell Acute Lymphoblastic Leukemia (T-ALL), B-cell Acute Lymphoblastic Leukemia (B-ALL), Acute Myeloid Leukemia (AML) and myelodysplastic syndromes. Solid tumors include carcinoma, sarcoma, or blastoma, and more preferably the bladder, brain, breast, uterus, oesophagus and liver cancers, colorectal cancer, renal cancer, melanoma, ovarian cancer, prostate cancer, pancreatic cancer and lung cancer, especially non-small-cell lung cancer and small-cell lung cancer.

‘Cₘₐₓ’ is the maximum (or peak) serum concentration that a drug achieves in a specified compartment or test area of the body after the drug has been administered and before the administration of a second dose.

The term ‘jointly therapeutically effective’ means that the therapeutic agents may be given separately (in a chronologically staggered manner, especially a sequence-specific manner) in such time intervals that they still show a (preferably synergistic) interaction (joint therapeutic effect) in the warm-blooded animal, especially human, to be treated. Whether this is the case can, inter alia, be determined by following the blood levels, showing that both compounds are present in the blood of the human to be treated at least during certain time intervals.

‘Synergistically effective’ or ‘synergy’ means that the therapeutic effect observed following administration of two or more agents is greater than the sum of the therapeutic effects observed following the administration of each single agent.

As used herein, the term ‘treat’, ‘treating’ or ‘treatment’ of any disease or disorder refers in one embodiment, to ameliorating the disease or disorder (i.e., slowing or arresting or reducing the development of the disease or at least one of the clinical symptoms thereof). In another embodiment ‘treat’, ‘treating’ or ‘treatment’ refers to alleviating or ameliorating at least one physical parameter including those which may not be discernible by the patient. In yet another embodiment, ‘treat’, ‘treating’ or ‘treatment’ refers to modulating the disease or disorder, either physically, (e.g., stabilization of a discernible symptom), physiologically, (e.g., stabilization of a physical parameter), or both.

As used herein, a subject is ‘in need of’ a treatment if such subject would benefit biologically, medically or in quality of life from such treatment.

In another aspect, provided is a method for sensitizing a human who is (i) refractory to at least one chemotherapy treatment, or (ii) in relapse after treatment with chemotherapy, or both (i) and (ii), wherein the method comprises administering a Bcl-2 inhibitor, which is Compound A, in combination with a hypomethylating agent, as described herein, to the patient. A patient who is sensitized is a patient who is responsive to the treatment involving administration of Compound A in combination with a hypomethylating agent, as described herein, or who has not developed resistance to such treatment.

‘Medicament’ means a pharmaceutical composition, or a combination of several pharmaceutical compositions, which contains one or more active ingredients in the presence of one or more excipients.

‘AML’ means acute myeloid leukemia.

‘Standard-of-care drug’ or ‘standard-of-care chemotherapy’ includes idarubicin, daunorubicin, mitoxantrone, cytarabine, decitabine, guadecitabine or azacitidine. Particularly, ‘standard-of-care drug’ or ‘standard-of-care chemotherapy’ means azacitidine.

In the pharmaceutical compositions according to the invention, the proportion of active ingredients by weight (weight of active ingredients over the total weight of the composition) is from 5 to 50%.

Among the pharmaceutical compositions according to the invention there will be more especially used those which are suitable for administration by the oral, parenteral and especially intravenous, per- or trans-cutaneous, nasal, rectal, perlingual, ocular or respiratory route, more specifically tablets, dragées, sublingual tablets, hard gelatin capsules, glossettes, capsules, lozenges, injectable preparations, aerosols, eye or nose drops, suppositories, creams, ointments, dermal gels etc.

The pharmaceutical compositions according to the invention comprise one or more excipients or carriers selected from diluents, lubricants, binders, disintegration agents, stabilisers, preservatives, absorbents, colorants, sweeteners, flavourings etc.

By Way of Non-Limiting Example there May be Mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The compounds of the combination may be administered simultaneously or sequentially. The administration route is preferably the intravenous infusion, and the corresponding pharmaceutical compositions may allow the instantaneous or delayed release of the active ingredients. The compounds of the combination may moreover be administered in the form of two separate pharmaceutical compositions, each containing one of the active ingredients, or in the form of a single pharmaceutical composition, in which the active ingredients are in admixture.

The useful dosage regimen varies according to the sex, age and weight of the patient, the administration route, the nature of the cancer and of any associated treatments and ranges from 12 mg to 1500 mg of Bcl-2 inhibitor (Compound A) per week, more preferably from 25 mg to 1000 mg per week. The dose of the hypomethylating agent, as described herein, will be the same as that used when it is administered on its own. In particular, azacitidine will be administered via subcutaneous (SC) injection or IV infusion at a dose of 75 mg/m² body surface area. Azacitidine will be administered at each cycle daily for 5-consecutive days (D1-D5) followed by a 2-day break (D6-D7) and then for 2 days (D8-D9) followed by a rest period of 19 days.

Pharmacological and Clinical Data

### Example 1: In Vitro Evaluation of the Growth Inhibition, Inhibition of Viability and Percentage of Apoptosis after Combinations of Compound A Treatment with 5-Azacitidine in AML Cell Lines (OCI-AML3, HL-60, MV4;11, EOL-1)

The combination of Compound A with 5-azacitidine was tested in four AML cell lines. Profiling for single agents was performed to select the appropriate dose range for the combination studies.

Cell viability, growth inhibition (GI) and % of apoptotic cells (not shown) were evaluated by fluorescence imaging assay (assay in which the cells are stained with Hoechst 34580 (Invitrogen, ref #H3570) and NucView NucView (VWR, ref #10403) probes, which is read by the OperaPhenix High Content Imaging platform).

The synergy effect was analysed with the Chalice software.

Material and Method

Cell lines were sourced and maintained in the basic media supplemented with FBS (Fetal Bovine Serum) as indicated in Table 1. In addition, all media contained penicillin (100 IU/mL), streptomycin (100 μg/mL) and L-glutamine (2 mM).

Cell lines were cultured at 37° C. in a humidified atmosphere containing 5% CO₂ and expanded in T-150 flasks. In all cases cells were thawed from frozen stocks, expanded through ≥1 passage using appropriate dilutions, counted and assessed for viability using a ViCell cell counter. All cell lines were determined to be free of mycoplasma contamination in-house. Stock solutions of compounds were prepared at a concentration of 5 mM in DMSO and stored at −20° C.

In order to analyse the activity of the compounds as single agents, AML cells were seeded in appropriate condition in 384 well plates, in 80 μL of culture medium. The incubation time (cells+test drug and Hoechst/NucView staining) lasted 96 h. Then, cells were treated for the 72 h time point either with vehicle only (DMSO) or with 9 different doses of Compound A in combination with 9 doses of azacitidine. Final concentration of DMSO was 0.2% in a final volume of 100 μL. One plate of non-treated AML cells was stained with 200 ng/mL of Hoechst and 10 μM of NucView during 3 hours in order to obtain a basal level of number of viable cells and apoptosis.

This plate containing cells were acquired with the Opera Phenix imaging system with the 5× objective and programmed at 37° C. and 5% CO₂.

Cells treated during 72 h were stained with 200 ng/mL of Hoechst and 10 μM of NucView then incubated at 37° C. during 3 h. Plates containing cells were acquired with the Opera Phenix imaging system with the 5× objective and programmed at 37° C. and 5% CO₂.

Effects of the compounds, as single agent or in combination, on cell viability were assessed after 3 days of incubation at 37° C./5% CO₂ by quantification of cellular ATP levels using CellTiterGlo at 75 μL reagent/well. All the experiments were performed in duplicates. Luminescence was quantified on a multipurpose plate reader. Single agent IC₅₀s were calculated using standard four-parametric curve fitting. IC₅₀ is defined as the compound concentration at which the CTG signal is reduced to 50% of that measured for the vehicle (DMSO) control (Table 2).

Potential synergistic interactions between compound combinations were assessed using the Excess Inhibition 2D matrix according to the Loewe additivity model and are reported as Synergy Score (Lehar et al., Nature Biotechnology 2009, 27(7), 659-66). All calculations were performed using Chalice™ Bioinformatics Software available in Horizon website.

The doubling time indicated in Table 1 is the mean of the doubling time obtained in the different passages (in T-150 flasks) performed from the thawing of the cells to their seeding in the 384-well plates.

Synergy Score

SS˜0→Additive

SS≥1→Weak Synergy

SS≥2→Synergy

TABLE 1
AML cell lines and their culture conditions used in the experiments.

| | | Cell line | | | Doubling |
|---|---|---|---|---|---|
| | Cell line | supplier | | | time |
| Cell line | supplier | reference | Origin | Culture medium | (hours) |
| OCI-AML3 | DSMZ | ACC 582 | Human | alpha-MEM medium | n1 = 30 |
| | | | | supplemented with 20% FBS | n2 = 31 |
| HL-60 | NA | NA | Human | IMDM medium supplemented | n1 = 35 |
| | | | | with 20% FBS | n2 = 38 |
| MV4; 11 | ATCC | CRM-9591 | Human | RPMI medium supplemented | n1 = 31 |
| | | | | with 20% FBS | n2 = 29 |
| EOL-1 | DSMZ | ACC 386 | Human | RPMI medium supplemented | n1 = 42 |
| | | Lot 14 | | with 10% FBS | n2 = 36 |

TABLE 2
Single agent IC₅₀ values for Compound A and 5-azacitidine
as well as the synergy scores for Compound A in combination
with 5-azacitidine in 4 AML cell lines are indicated. Interactions
were deemed synergistic when scores ≥2.0 where observed.
Start concentrations of compounds and mean of max inhibition
of the synergy scores are also documented.

| | Compound A | | 5-azacitidine | | |
|---|---|---|---|---|---|
| | Start | | Start | | Combination |
| | conc | IC₅₀ | conc | IC₅₀ | Mean of Synergy |
| Cell Lines | [μM] | [μM] | [μM] | [μM] | Score |
| OCI-AML3 | 10 | 5.689 | 10 | 7.103 | 17.3 |
| HL-60 | 10 | 3.099 | 10 | >10 | 19.4 |
| MV4; 11 | 0.1 | 0.003 | 10 | 0.689 | 32.9 |
| EOL-1 | 0.1 | 0.018 | 10 | 0.187 | 12.5 |

Results

The effect on proliferation of combining Compound A, the Bcl-2 inhibitor according to the invention, with azacitidine was assessed in a panel of 4 AML cell lines. In combination with azacitidine, synergistic growth inhibition (i.e. Synergy Scores above 2 (Lehar et al, 2009)) for all the cell lines tested was observed (Table 2). These data indicate that the combination of Compound A with azacitidine enhance the anti-proliferative effect of each single agent and thus could provide benefit to the treatment of AML patients.

### Example 2: Clinical Trial Protocol

A phase I/II, open label, dose escalation part (phase I) followed by non-comparative expansion part (phase II), multi-centre study, evaluating safety, pharmacokinetics and efficacy of Compound A, a Bcl2 inhibitor combined with azacitidine in adult patients with previously untreated acute myeloid leukemia not eligible for intensive treatment.

Primary Objectives:
Phase 1/Dose escalation part:
   To determine the safety profile, the tolerability and the Recommended Phase II Dose (RP2D) of Compound A combined to azacitidine
Phase 2/For both expansion parts:
   To evaluate the efficacy of Compound A combined to azacitidine as measured by Complete Response (CR) rate

Secondary Objectives:
Phase 1/Dose escalation part:
   To determine the PharmacoKinetics (PK) profile of Compound A and azacitidine administered in combination and of potential metabolite(s) (if applicable)
   To evaluate the efficacy of Compound A combined to azacitidine
Phase 2/For both expansion parts:
   To evaluate the efficacy of Compound A combined to azacitidine as measured by Overall Response Rate (ORR), CR rate by initiation of cycle 2 (CR2), Complete Response rate with incomplete blood recovery (CRi rate), Duration of Response (DOR), Event Free Survival (EFS), Progression Free Survival (PFS), Overall Survival (OS), and time to first response
   To evaluate the depth of response and the duration of response by analysing the Minimal Residual Disease (MRD) of participants with CR
   To determine the safety profile and tolerability of Compound A combined to azacitidine
   To further characterised the PK profile of Compound A and azacitidine administered in combination and of potential metabolite(s) (if applicable)

Test Drug:

Compound A (test drug) is combined to azacitidine. During dose escalation, only Compound A dose will escalate.

Dose Allocation Methodology for Compound A:

In the first cohort, the dose allocation will start at the weekly dose of 50 mg (from Cycle 1 Day 8 abbreviated as C1D8) with 2 ramp up doses of 25 mg. Participants will be included in cohorts of 3 to 6 evaluable participants.

A panel of Compound A doses from 12 mg to 450 mg could be tested according to the dose allocation process of the Bayesian Logistic Regression Model. Intermediate dose level could be tested during the study. During end of cohort meetings, ramp up doses and full dose of Compound A could be adapted according to available safety, pharmacokinetic and efficacy results. Doses over 450 mg could be tested if needed.

No dose reduction below level 12 mg is permitted for this study.

Cohorts may be added at any dose level below the Maximum Tolerated Dose (MTD) in order to better understand safety and PK.

After the MTD (if characterized) and RP2D have been determined, 3 cohorts of up to 6 patients no higher than the MTD may be allowed at various dose levels for PK/PD (PharmacoKinetics/PharmacoDynamics) purposes without Dose Limiting Toxicity (DLT) evaluation.

Dose and Treatment Schedule:

Compound A will be administered via intravenous (IV) infusion over 30 min (+/−5 min) via central or peripheral venous line.
Solution for infusion will be prepared using a 20 mL vials containing 150 mg of Compound A (expressed as free base) formulated with a HP-β-cyclodextrin as described below.

Duration of infusion of Compound A could be adapted based on preliminary safety and PK data.

Whatever the phase of the study, azacitidine will be administered via subcutaneous (SC) or IV infusion at a dose of 75 mg/m² body surface area.

On days of concomitant treatment, Compound A should be given first, azacitidine will be given between 30 minutes and 1 hour later (respecting a delay of a minimum of 30 minutes and not exceeding 1 hour between end of infusion of Compound A and beginning of administration of azacitidine).

Administration Schedule 1: Combination Treatment During Phase 1 Part:

Ramp Up Dose Period:

2 ramp-up infusions of Compound A will be administered to participant on C1D-4 or C1D-3 (3 days or 4 days before starting the combination therapy) and on C1D1 (combined with first administration of azacitidine). This dose could be adapted during end of cohort meetings. The first dose of ramp-up will be given before starting the combination with azacitidine. The second ramp-up dose will be co-administered with azacitidine.

A Treatment Cycle Will Consist of 28 Days:
Compound A full dose will be administered on a 4-week cycle (Day1 abbreviated as D1, D8, D15, D22). On cycle 1, the C1D1 will corresponds with the second ramp-up dose.
Azacitidine will be administered at each cycle daily for 5-consecutive days (D1-D5) followed by a 2-day break (D6-D7) and then for 2 days (D8-D9) followed by a rest period of 19 days.

Administration Schedule 2: Combination Treatment During Phase 1 Part:

Ramp Up Dose Period

One ramp up infusion of Compound A will be administered to participant on C1D-4 or C1D-3. This dose could be adapted during end of cohort meetings. Only 1 ramp up dose will be given to participants in this administration schedule 2.

Each Treatment Cycle Will Consist of 28 Days:
Week 1:
   Compound A full dose will be administered on D1, D3 and D5.
   Azacitidine will be administered for 5-consecutive days (D1-D5) followed by a 2-day break (D6-D7).
Week 2:
   Compound A full dose will be administered on D8.
   Azacitidine will be administered for 2-consecutive days (D8-D9) followed by a rest period of 19 days.

Three additional doses of Compound A may be added on D2, D4 and D9 according to decision during End of Cohort meeting as further described below.

Administration Schedule 3: Combination Treatment During Phase 1 Part:

Ramp Up Dose Period

One ramp up infusion of Compound A will be administered to participant on C1D-4 or C1D-3. This dose could be adapted during end of cohort meetings. Only 1 ramp up dose will be given to participants in this administration schedule 3.

Each Treatment Cycle Will Consist of 28 Days:
Week 1:
   Compound A full dose will be administered on D1, D2, D3, D4 and D5.
   Azacitidine will be administered for 5-consecutive days (D1-D5) followed by a 2-day break (D6-D7).
Week 2:
   Compound A full dose will be administered on D8 and D9.
   Azacitidine will be administered for 2-consecutive days (D8-D9) followed by a rest period of 19 days.

Combination Treatment During Phase II Part:

Ramp up dose and full dose will be the RP2D determined during phase I part.

Ramp Up Dose Period

2 ramp-up infusions of Compound A will be administered to participant on D-4 or D-3 and on D1. The first dose of ramp-up will be given before starting the combination with azacitidine. The second ramp-up dose will be co-administered with azacitidine.

A Treatment Cycle Will Consist of 28 Days:
Compound A full dose will be administered on a 4-week cycle (D1, D8, D15, D22). On cycle 1, the C1D1 will corresponds to the second ramp-up dose.
Azacitidine will be administered at each cycle daily for 5-consecutive days (D1-D5) followed by a 2-day break (D6-D7) and then for 2 days (D8-D9) followed by a rest period of 19 days.

Alternatively, the administration schedule to be used during the phase II part will be determined during last end of cohort meeting of phase I part and will be implemented via an amendment to the clinical study protocol.

Preparation of Lyophilisates of Compound a Solubilised in a HP-β-Cyclodextrin in 20 mL Vials:

The lyophilisates are prepared in 20 mL vials in which it will be possible to reconstitute the solution to be administered by the parenteral route. They are obtained by lyophilisation of a 20% Cavitron™ W7HP5 solution containing a dose of 20 mg/mL of Compound A (free base).

Procedure

In a 5 L reactor, weigh 1500 g of water. With magnetic stirring, create a vortex and then pour in 600 g of Cavitron™ W7HP5. Stir the medium at ambient temperature until the cyclodextrin is solubilised completely, and add 68.16 g of ‘Compound A, H₂SO₄’ and heat the solution to not more than 60° C. Place the suspension under magnetic stirring for several hours and then allow the medium to return to a temperature below 30° C. Measure the pH of the solution so obtained, then adjust it to pH 3.0 with 0.5M NaOH solution poured slowly. Make up the solution to a volume of 3 L by adding water, while maintaining magnetic stirring.

Pass the solution so obtained through a 0.2 μm filter.

Fill the 20 mL vials with the filtered solution so that each vial contains at least 150 mg of Compound A (expressed as free base) and subject the samples to a lyophilisation step.

The resulting lyophilisate is intended to be used for the preparation of a pharmaceutical composition for parenteral administration.

Methodology:

The phase I/II non-comparative study, open-label, multi-centre study is divided into 2 phases:
A single arm dose escalation phase I part:

An adaptive Bayesian Logistic Regression Model (BLRM) guided by an escalation with overdose control (EWOC) method designed with dose-escalating levels, will be used to make dose recommendations based on the occurrence of DLT(s) until the end of cycle 1 and estimate the MTD (if characterized)/RP2D for Compound A combined to azacitidine.

After the RP2D is determined, 3 cohorts of up to 6 participants can be added at any dose level at or below the MTD (if characterized) in order to better understand safety, PK and PD, without DLT evaluation.
Dose expansion phase II part:
First expansion:

A Bayesian 2-stage adaptive model with one futility interim analysis designed in one arm at the RP2D will be conducted after the dose escalation part.

During stage 1, participants will be enrolled and treated at the corresponding RP2D. At the end of stage 1, a bayesian futility interim analysis will be performed in 10 participants by initiation of cycle 2 or early discontinued.

According to the futility interim analysis result at the end of stage 1, recruitment could be:
Stopped, if results on CR rate by initiation of cycle 2 are considered futile;
Continued if results on CR rate by initiation of cycle 2 are considered not futile. In that case, one additional cohort of 11 participants will be enrolled in stage 2 and treated at the corresponding RP2D until the predefined end of study.

At end of stage 2, results on the overall CR rate considering participants included in stage 1 and stage 2 (approximately 21 participants) with at least 2 cycles or early discontinued will be provided.
Second expansion:

If early signs of strong activity in the escalation and/or first analysis of the first expansion, approximately 21 additional participants could be included to evaluate the combination activity in the TP53 sub-population with the same design as the initial expansion cohort.

For each expansion cohort, if there is strong evidence of activity at the end of stage 2 (based on the evaluation of 21 participants) and considering a global assessment of the safety, PK and PD data, up to 40 additional participants could be included to confirm the finding and increase the accuracy of the estimation following a consultation with Health Authorities.

1-30. (canceled)

31. A combination comprising:
(a) a Bcl-2 inhibitor which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (‘Compound A’): 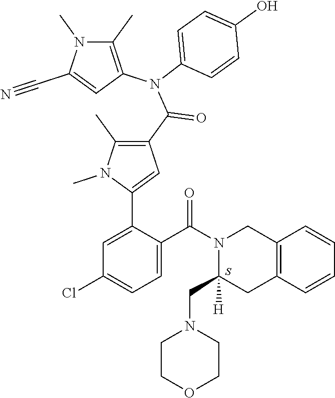
and
(b) a hypomethylating agent selected from decitabine, azacitidine and guadecitabine, for simultaneous, sequential or separate use.

32. The combination according to claim 31, wherein the hypomethylating agent is azacitidine.

33. The combination according to claim 31, wherein Compound A is in the form of a hydrogen sulfate salt.

34. A method of treating cancer in a subject in need thereof, comprising administration of the combination according to claim 31.

35. The method according to claim 34, wherein the cancer is a haematological malignancy.

36. The method according to claim 35, wherein the haematological malignancy is acute myeloid leukemia (AML).

37. The method according to claim 35, wherein the haematological malignancy is myelodysplastic syndromes.

38. The method according to claim 35, wherein the haematological malignancy is lymphoma.

39. The method according to claim 35, wherein the haematological malignancy is chronic lymphocytic leukemia.

40. The method according to claim 35, wherein the haematological malignancy is multiple myeloma.

41. The method according claim 34, wherein Compound A and the hypomethylating agent are provided in amounts which are jointly therapeutically effective for the treatment of cancer.

42. The method according to claim 34, wherein Compound A and the hypomethylating agent are provided in amounts which are synergistically effective for the treatment of cancer.

43. The method according to claim 42, wherein the Compound A and the hypomethylating agent are provided in synergistically effective amounts which enable a reduction of the dose required for each compound in the treatment of cancer, whilst providing an efficacious cancer treatment, with eventually a reduction in side effects.

44. The method according to claim 34, wherein Compound A is administered parentally, including intravenously.

45. The method according to claim 44, wherein the dose of Compound A per administration is from 25 mg to 1000 mg.

46. The method according to claim 45, wherein Compound A is administered once a week.

47. The combination according to claim 31, further comprising one or more excipients.

48. A pharmaceutical composition containing, separately or together,
(a) a Bcl-2 inhibitor which is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (‘Compound A’): 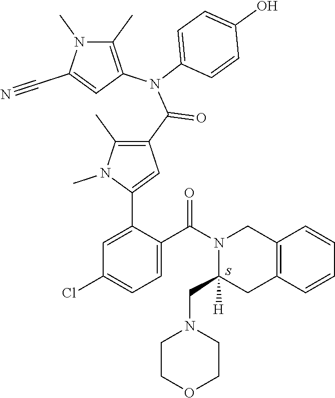
and
(b) a hypomethylating agent,
for simultaneous, sequential or separate administration, and wherein the Compound A and the hypomethylating agent are provided in effective amounts for the treatment of cancer.

49. The pharmaceutical composition according to claim 48, wherein the hypomethylating agent is azacitidine.

50. The method according to claim 41, wherein the hypomethylating agent is azacitidine.

51. The method according to claim 50, wherein Compound A and azacitidine are administered during a 28-day cycle as follows:
(i) Compound A is administered on day 1 (D1), day 8 (D8), day 15 (D15) and day 22 (D22) and,
(ii) azacitidine is administered according to a 5-2-2 schedule:
5-consecutive days (D1-D5) followed by a 2-day break (D6-D7) and then for 2 days (D8-D9),
followed by a rest period of 19 days.

52. The method according to claim 50, wherein Compound A and azacitidine are administered during a 28-day cycle as follows:
(i) Compound A is administered on day 1 (D1), day 3 (D3), day 5 (D5) and day 8 (D8) in the two first weeks of the cycle, wherein the single doses administered on D1, D3, D5 and D8 are identical to each other;
(ii) azacitidine is administered according to a 5-2-2 schedule:
5-consecutive days (D1-D5) followed by a 2-day break (D6-D7) and then for 2 days (D8-D9),
followed by a rest period of 19 days.

53. The method according to claim 50, wherein Compound A and azacitidine are administered during a 28-day cycle as follows:
(i) Compound A is administered on day 1 (D1), day 2 (D2), day 3 (D3), day 4 (D4), day 5 (D5), day 8 (D8) and day 9 (D9) in the two first weeks of the cycle, wherein the single doses administered on D1, D2, D3, D4, D5, D8 and D9 are identical to each other;
(ii) azacitidine is administered according to a 5-2-2 schedule:
5-consecutive days (D1-D5) followed by a 2-day break (D6-D7) and then for 2 days (D8-D9),
followed by a rest period of 19 days.

54. A method for sensitizing a patient who is (i) refractory to at least one chemotherapy treatment, or (ii) in relapse after treatment with chemotherapy, or both (i) and (ii), wherein the method comprises administering a jointly therapeutically effective amount of 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide (‘Compound A’): 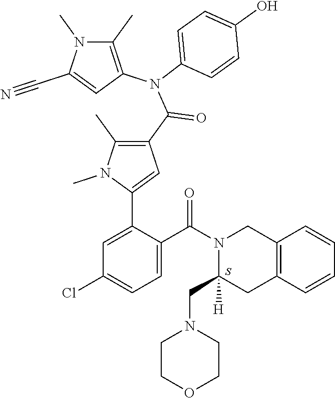
in combination with a hypomethylating agent, to said patient.

55. The method according to claim 54, wherein the hypomethylating agent is azacitidine.

56. The method according to claim 55, wherein Compound A and azacitidine are administered during a 28-day cycle as follows:
(iii) Compound A is administered on day 1 (D1), day 8 (D8), day 15 (D15) and day 22 (D22) and,
(iv) azacitidine is administered according to a 5-2-2 schedule:
5-consecutive days (D1-D5) followed by a 2-day break (D6-D7) and then for 2 days (D8-D9),
followed by a rest period of 19 days.

57. The method according to claim 55, wherein Compound A and azacitidine are administered during a 28-day cycle as follows:
(i) Compound A is administered on day 1 (D1), day 3 (D3), day 5 (D5) and day 8 (D8) in the two first weeks of the cycle, wherein the single doses administered on D1, D3, D5 and D8 are identical to each other;
(ii) azacitidine is administered according to a 5-2-2 schedule:
5-consecutive days (D1-D5) followed by a 2-day break (D6-D7) and then for 2 days (D8-D9),
followed by a rest period of 19 days.

58. The method according to claim 55, wherein Compound A and azacitidine are administered during a 28-day cycle as follows:
(i) Compound A is administered on day 1 (D1), day 2 (D2), day 3 (D3), day 4 (D4), day 5 (D5), day 8 (D8) and day 9 (D9) in the two first weeks of the cycle, wherein the single doses administered on D1, D2, D3, D4, D5, D8 and D9 are identical to each other;
(ii) azacitidine is administered according to a 5-2-2 schedule:
5-consecutive days (D1-D5) followed by a 2-day break (D6-D7) and then for 2 days (D8-D9),
followed by a rest period of 19 days.
